(19) United States
(12) Patent Application Publication
(10) Pub. No.: US 2017/0173345 A1
(43) Pub. Date: Jun. 22, 2017

(54) Multi-Tiered Wireless Powering System for Long-Term Implantable Medical Devices

(71) Applicants: THE REGENTS OF THE UNIVERSITY OF CALIFORNIA, Oakland, CA (US); U.S. Department of Veterans Affairs, Washington, DC (US)

(72) Inventors: Parinaz Abiri, Oakland, CA (US); Tzung K. Hsiai, Oakland, CA (US); Ahmad Abiri, Oakland, CA (US)

(21) Appl. No.: 15/383,973

(22) Filed: Dec. 19, 2016

Related U.S. Application Data
(60) Provisional application No. 62/269,680, filed on Dec. 18, 2015.

(51) Int. Cl. A61N 1/378; H02J 7/02; A61B 5/01; A61N 1/39; A61N 1/36; A61B 5/0402; H02J 50/10; A61N 1/362

(52) U.S. Cl. CPC A61N 1/3787; H02J 50/10; H02J 7/025; A61N 1/362; A61N 1/3956; A61N 1/3605; A61B 5/0402; A61B 5/01; A61B 2560/0219; A61N 1/0534

(57) ABSTRACT

A wireless powering system for an implantable medical device includes a first unit having a power source and an inductive coil. A second unit has an energy storage component and in alternate embodiments one or two inductive coils. The implantable medical device includes an inductive coil and a functional load. A method of wirelessly powering an implantable medical device is also included. The embodiments provide a method for wireless powering of an implantable medical device. Through the use of a three tiered inductive power system, power can be wirelessly transmitted from near the skin of the patient to the location of the implant. The implant can be reduced in size and can have a longer lifespan due to the elimination of an integrated battery. The versatility of the implant can allow for improved and more secure fixation to the tissue of interest.

### CROSS-REFERENCE TO RELATED APPLICATIONS

This application claims priority to U.S. provisional application No. 62/269,680 filed on Dec. 18, 2015 incorporated herein by reference in its entirety.

### STATEMENT REGARDING FEDERALLY SPONSORED RESEARCH OR DEVELOPMENT

This work was supported by the U.S. Department of Veterans Affairs, and the Federal Government has certain rights in the invention.

### BACKGROUND OF THE INVENTION

For decades, the primary method of non-pharmacologist therapy for patients suffering from bradyarrhythmias and conduction disorders has been through the use of implantable cardiac pacing devices. In 2009, there were greater than 230,000 pacemaker implants in the United States alone (see Mond, Harry G. et al. The 11th World Survey of Cardiac Pacing and Implantable Cardioverter-Defibrillators: Calendar Year 2009—A World Society of Arrhythmia's Project. (2011). Pacing and Clinical Electrophysiology 34(8): 1013-027.). Despite advancements in pacemaker technology, there still exist a large number of limitations in these devices that has resulted in damage to patient health and safety.

As illustrated in the example shown in FIG. 1A, current market-released pacemaker systems consist of a pulse generator, often placed subcutaneously or submuscular, and pacing leads, which travel from the pulse generator through nearby prominent veins (commonly the subclavian vein) and are inserted into the heart endocardium and/or placed into the coronary veins (see for example, Kirk, M. (2006) Basic Principles of Pacing, in Implantable Cardiac Pacemakers and Defibrillators: All You Wanted to Know (eds A. W. C. Chow and A. E. Buxton), Blackwell Publishing Ltd, Oxford, UK). Although complications can occur at the site of the pocket in which the pulse generator is located, the most common problems associated with pacemaker implantation are lead-related complications (see Kirkfeldt, R. E. et al. (2014). Complications after cardiac implantable electronic device implantations: an analysis of a complete, nationwide cohort in Denmark. European Heart Journal, 35(18), 1186-1194.). These include lead dislodgement, myocardial perforation during lead placement, extracardiac stimulation from microdislodgment, symptomatic venous stenosis in the presence of multiple pacemaker leads, cardiac tamponade and pericarditis, twiddler syndrome, valvular damage from lead constriction, vascular occlusion and hemorrhaging from conductor coil fracture, and electrical abnormalities from insulation break. (See Udo, Erik O. et al. (2012). Incidence and Predictors of Short- and Long-term Complications in Pacemaker Therapy: The FOLLOWPACE Study. Heart Rhythm 9(5): 728-35. See Enes Elvin Gul et al. (2011). Common Pacemaker Problems: Lead and Pocket Complications, Modern Pacemakers—Present and Future, Prof. Mithilesh R Das (Ed.), ISBN: 978-953-307-214-2, InTech, DOI: 10.5772/12965.) X-ray images of fractured leads are shown in FIGS. 1B and 1C. Overall, about 10% of worldwide pacemaker implants are associated with lead-related problems (see Mela, Theofanie et al. (2015). Leadless Pacemakers: Leading Us into the Future. Eur Heart J European Heart Journal. 36(37): 2520-522.). It is also important to note that once a damaged lead is discovered, removal of the leads can prove extremely dangerous and at times impossible due to fibrosis that strongly attaches the device to the surrounding tissue (see Hauser, R. G. et al. (2009). Deaths and Cardiovascular Injuries Due to Device-assisted Implantable Cardioverter-defibrillator and Pacemaker Lead Extraction. Europace 12(3): 395-401.). Often, additional leads are simply added without the removal of the previous devices, further risking patient safety.

To combat these limitations, medical device manufacturers have developed the leadless cardiac pacemaker systems (see Mela et al.). These devices consist of a small package containing the pacemaker electrode, logic circuitry, and a small battery, a system that is then fixated into the heart endocardium. In one example, the device is about 2.5 cm in length and 0.7 cm in diameter. It is implanted via catheter delivery and lodged into the endocardium using four nitinol tines. The challenges facing this leadless device include a difficult procedural fixation mechanism with various mechanical failure points, the large size of the delivery catheter that can lead to vascular complications, significantly longer fluoroscopy time for implantation, limited battery lifetime, and the limited ability to perform only single-chamber pacing. In another example, the device is about 4 cm in length and 0.6 cm in diameter. Though delivered via a smaller catheter, it still faces many of the limitations described above. In addition, the device, which achieves fixation using a distal non-retractable, single-turn (screw-in) steroid-eluting helix, has experienced multiple myocardial perforations and patient deaths during clinical trials.

As both of these devices are yet to be market-released, there is currently no long-term data on possible device migration, dislodgement, and electrical connection issues. Due to the reliance of the method of fixation on small tines or a screw-in helix inside a beating chamber and against extreme pressure gradients, mechanical damage can occur depending on angle of implantation, stability of position, number of retractions during deployment, and other patient-specific variables. Furthermore, device dependence on a limited lifetime battery introduces a problem with long-term implants. With the expiration of the battery, a second device would need to be deployed while the original remains in place due to extensive surrounding fibrosis.

Many of the complications associated with both of the described leadless devices are due to their use of an attached single-use limited lifetime battery for power delivery. Wirelessly powered devices have been under extensive study. However, power dissipation over long distances (e.g. >5 cm) as well as tissue absorption loss and heating have significantly impaired the ability to inductively power medical devices.

What is needed in the art is an improved wireless transfer mechanism that can achieve power transfer over long distances within safe absorption limits while accommodating for the small anatomical real estate available within the body.

### SUMMARY OF THE INVENTION

In one embodiment, a wireless powering system for an implantable medical device including a first unit including a first power source electrically coupled to a first inductive coil; a second unit including a first energy storage component electrically coupled to a second inductive coil; and a third unit including a third inductive coil electrically coupled to a functional load. In one embodiment, the second unit further includes a fourth inductive coil, wherein the first inductive coil is configured to transfer power to the second inductive coil at a first frequency, and the fourth inductive coil is configured to transfer power to the third inductive coil at a second frequency. In one embodiment, the first frequency is lower than the second frequency. In one embodiment, the first and second frequency are substantially the same. In one embodiment, the first inductive coil is larger than the second, third and fourth inductive coils. In one embodiment, the third inductive coil is smaller than the first, second and fourth inductive coils. In one embodiment, the first unit is configured for external attachment to a patient's body. In one embodiment, the first unit is implantable. In one embodiment, the first power source is a battery or a capacitor. In one embodiment, the first power source is a rechargeable battery. In one embodiment, the first energy storage component is a second battery that is rechargeable. In one embodiment, the first energy storage component is a capacitor. In one embodiment, the second unit further includes a logic unit electrically coupled to the first energy storage component and configured to receive data from the third unit. In one embodiment, the logic unit is configured to transmit data to the third unit based on the received data. In one embodiment, the transmitted data includes an instruction to adjust a function of the functional load. In one embodiment, the function is a stimulatory or sensing function. In one embodiment, the logic unit is an integrated circuit. In one embodiment, the third unit includes a sensor configured to provide feedback data to at least one of the functional load, the first unit and the second unit. In one embodiment, the functional load is one of a sensor, electrode, actuator, motor and valve. In one embodiment, the functional load is one of a measuring device, sensing device, actuating device, stimulation device, and therapeutic device. In one embodiment, the functional load is one of a pacemaker, pressure sensor, temperature sensor, flow sensor, flow pump, implantable cardioverter defibrillator, ECG device, deep brain stimulator, and neuromodulator. In one embodiment, the third unit includes a second energy storage component electrically coupled to the third inductive coil and the functional load. In one embodiment, the third unit is configured to continuously receive power from the second unit. In one embodiment, the third unit is configured to intermittently receive power from the second unit. In one embodiment, the third unit includes a sensor configured to provide feedback data to at least one of the functional load, the first unit and the second unit. In one embodiment, the second unit includes a sensor configured to provide feedback data to at least one of the first unit and the second unit. In one embodiment, the third unit consists of a third inductive coil electrically coupled to a functional load.

In one embodiment, a method of wirelessly powering an implantable medical device includes the steps of transferring power from a first unit to a second unit through induction at a first frequency; and transferring power from the second unit to a third unit through induction at a second frequency. In one embodiment, the first frequency is lower than the second frequency. In one embodiment, the first and second frequency are substantially the same. In one embodiment, the first unit includes a first inductive coil, the second unit includes a second inductive coil, and the third unit includes a third inductive coil. In one embodiment, the first inductive coil is larger than the second, third and third inductive coils. In one embodiment, the second unit includes a fourth inductive coil, and wherein the third inductive coil is smaller than the first, second and fourth inductive coils. In one embodiment, the method includes the step of attaching the first unit to a patient's body. In one embodiment, the method includes the step of subcutaneously implanting the first unit in a patient's body. In one embodiment, the method includes the step of implanting the second unit in a thoracic cavity of a patient. In one embodiment, the method includes the step of implanting the third unit in contact with the heart of a patient. In one embodiment, the method includes the step of transmitting sensory data from the third unit to the second unit. In one embodiment, the method includes the step of transmitting functional data from the second unit to the third unit and adjusting the function of the third unit based on the functional data. In one embodiment, the method includes the step of transmitting feedback data from the third unit to at least one of the functional load, the first unit and the second unit. In one embodiment, the method includes the step of continuously transferring power from the second unit to the third unit. In one embodiment, the method includes the step of intermittently transferring power from the second unit to the third unit. In one embodiment, the method includes the step of sensing feedback including at least one of medical device function feedback or patient physiological feedback from a sensor at the second unit, and transferring power from the second unit to the third unit based on the sensed feedback.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1A is a prior art diagram of a conventional pacemaker implant, and FIGS. 1B and 1C are prior art X-ray images of fractured pacemaker leads.

FIG. 2A is a diagram illustrating the components of a system including a first unit, a second unit and a third unit, where a sensor is part of the third unit according to one embodiment, and FIG. 2B is a diagram illustrating the components of a system including a first unit, a second unit and a third unit, where the sensor is part of the second unit, and the third unit operates strictly to receive power and activate the element that provides end use medical functionality to the patient, according to one embodiment.

FIGS. 3 and 4 are diagrams illustrating the locational relationship between components of the system and the skin, the thoracic cavity and the heart according to one embodiment. In this embodiment, the first unit is positioned outside the body at the surface of the patient's skin.

FIG. 5 is a diagram illustrating the locational relationship between components of the system and the skin, the thoracic cavity and the heart according to one embodiment. In this embodiment, the first unit is subcutaneously implanted.

FIG. 6 is a diagram illustrating electrode placement on a heart according to one embodiment.

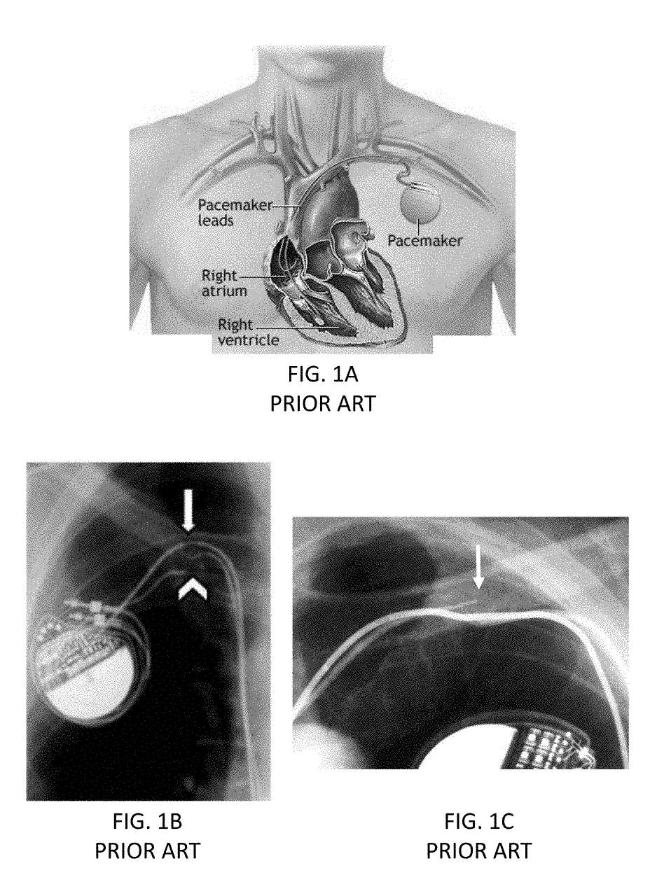

### DETAILED DESCRIPTION OF THE INVENTION

It is to be understood that the figures and descriptions of the present invention have been simplified to illustrate elements that are relevant for a more clear comprehension of the present invention, while eliminating, for the purpose of clarity, many other elements found in systems and methods of wirelessly powering implanted medical devices. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Ranges: throughout this disclosure, various aspects of the invention can be presented in a range format. It should be understood that the description in range format is merely for convenience and brevity and should not be construed as an inflexible limitation on the scope of the invention. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Referring now in detail to the drawings, in which like reference numerals indicate like parts or elements throughout the several views, in various embodiments, presented herein is a system and method for wirelessly powering an implantable medical device.

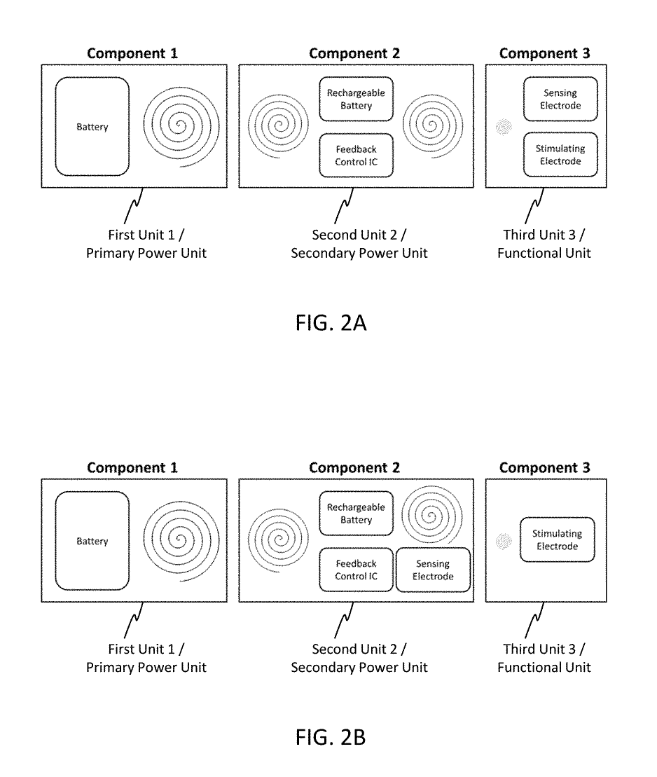

A system for the wireless transfer of power in implantable medical devices is described. In one embodiment, the system includes of at least three distinctly packaged components, as illustrated in FIGS. 2A and 2B.

With reference first to FIG. 2A, the first unit 1, which acts at least in part as the primary power unit is the primary power source. It includes at least one battery that delivers charge to at least one inductive coil. In one embodiment, the primary power unit 1 is placed external to the patient (see FIG. 4), for example in the form of a chest strap. In another embodiment, the primary power unit 1 is placed inside the body (see FIG. 5), such as subcutaneously or submuscular. The primary power unit 1 may be replaced or charged over a span of seconds, minutes, hours, days, weeks, months, or years. The inductive coil in the primary power unit 1 may be of any size, but in the preferred embodiment, the inductive coil of the primary power unit 1 is the largest of the three components' inductive coils due to available real estate and to increase efficiency of power transfer. Additionally, in one embodiment, the primary power unit 1 includes other diagnostic, sensing, monitoring, measuring, or therapeutic components.

The second unit 2, which acts at least in part as the secondary power unit mainly functions as a secondary power unit or source with the possibility of additional computational functionalities. It includes at least one inductive coil that receives power from the primary component, at least one battery or capacitor that charges using power from the at least one inductive coil receiver and delivers charge to at least one inductive coil transmitter. In one embodiment, the battery is a rechargeable battery that can be charged intermittently by the primary power unit 1 and the primary power unit 1 is physically removed or electrically turned off until the next charging cycle. In another embodiment, the secondary power unit 2 continuously receives power from the primary power unit 1. In one embodiment, the secondary power unit 2 is a flexible circuit that can adapt to anatomical constraints. In another embodiment, the secondary power unit 2 is a partially flexible device that can be inserted into the body using minimally invasive techniques. In another embodiment, the secondary power unit 2 does not contain flexible parts but is small enough to be inserted into the body using minimally invasive techniques. The inductive coil in the secondary power unit 2 may be any size, but in the preferred embodiment, the inductive coil of the secondary power unit 2 is smaller than the primary power unit 1's inductive coil, and significantly larger than the inductive coils of the third unit 3 due to available real estate and to increase efficiency of power transfer. The secondary power unit 2 can be positioned any distant between the first and third component, however, in the preferred embodiment, the secondary power unit 2 is positioned closer to the third unit 3 due to the ability to achieve more efficient coupling between the first and second component's inductors. In the preferred embodiment, the secondary power unit 2 also contains a controller or logic unit, such as an integrated circuit (IC), that receives telemetry data from the third unit 3, performs analysis, and provides feedback to the third unit 3 to adjust stimulatory or sensing function, thus performing primary device functional computations to minimize power requirements of the third unit 3. In one embodiment, the secondary power unit 2 transmits telemetry data to the primary power unit 1 or to another device external to the patient.

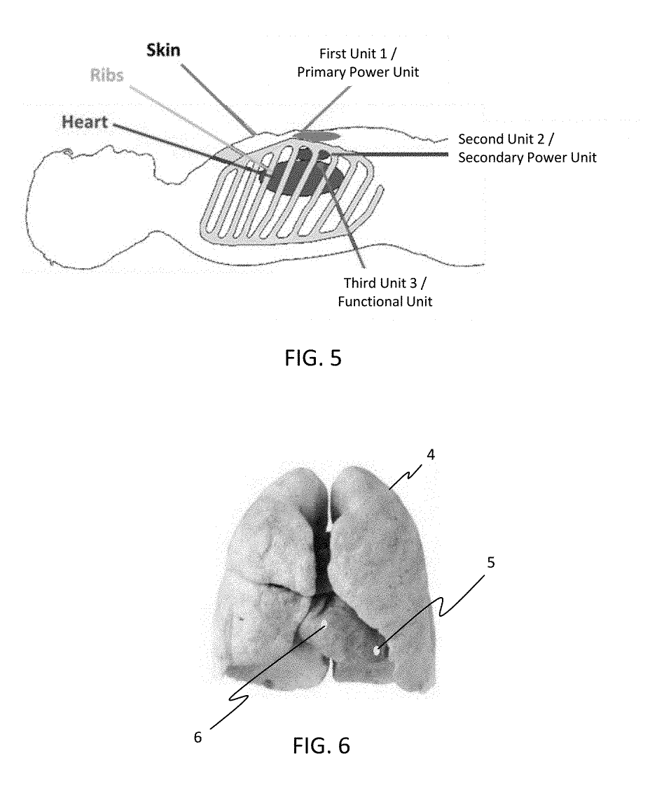

The third unit 3 includes the functional component of the medical device, and it performs the primary treatment function of the system, for example functioning as a cardiac pacer. It includes at least one inductive coil that receives power from the secondary power unit 2 and delivers power to a functional load, for example an electrode 5 that stimulates cardiac muscle tissue and a sensor 6 that detects cardiac muscle electrical activity in the heart 4, as shown in FIG. 6. In one embodiment, the third unit 3 includes at least one stimulator. In one embodiment, the third unit 3 includes at least one sensor. As contemplated herein, the sensor can be a sensing electrode, a physiological sensor, a sensor for detecting medical device performance, or other similar types of sensors known in the art. In one embodiment, the sensor is integrated into the housing of the third unit 3. In one embodiment, the sensor is separate from and connected to the housing of the third unit 3, such as by for example a wire that communicates the signal detected from the sensor back to the third unit 3. In one embodiment, the third unit 3 may also include a battery or capacitor. In another embodiment, the third unit 3 may also include a feedback control system that adjusts the function of the third unit 3. In one embodiment, the third unit 3 continuously receives power from the secondary power unit 2. In the preferred embodiment, the third unit 3 receives power from the secondary power unit 2 intermittently only when stimulation or sensing is scheduled. The inductive coil in the third unit 3 may be any size, but in the preferred embodiment, the inductive coil of the third unit 3 is the smallest of the three components' inductive coils to allow a more secure, safer, and versatile fixation mechanism, for example via an integrated stent placed in the organ's vessels, direct injection into tissue, or mechanically stabilized into tissue via tertiary fixation techniques.

In one embodiment, with reference not to FIG. 2B, the third unit 3 is stripped of at least one communication, controller or sensing functionality, which is moved instead to the second unit 2. In one embodiment, the third unit 3 is stripped of all communication, controller or sensing functionality, which is moved instead to the second unit 2. This way, the third unit 3 is strictly a unit that receives power for activating the component that provides end use medical functionality to the patient (e.g. a stimulation electrode, a fluid flow pump, etc.), without any additional functionality. Advantageously, this most simplified form of the third unit 3 allows for minimizing its size and geometry while maximizing its placement options. Further, the simplified form of the third unit is more reliable since it contains a minimal number of components. This also allows for intermediate units that are easier to access (e.g. the second unit 2) and have more components to be replaced if needed, without requiring access to units (e.g. the third unit 3) that are more deeply implanted and positioned closer to vital organs. In one embodiment, the second unit 2 includes a controller for the functional component on the third unit 3 that performs the primary treatment function. For example, the second unit 2 can include a sensing electrode that provides feedback for controlling a stimulating electrode on the third unit 3. In one embodiment, the sensor is integrated into the housing of the second unit 2. In one embodiment, the sensor is separate from and connected to the housing of the second unit 2 by a wire. In one embodiment, the component that performs the primary treatment in the third unit 3 is directly activated by power received from the second unit 2. Accordingly, in certain embodiments the treatment component is activated as a direct function of the intensity and timing of power transfer from the second unit 2. In one embodiment, power between the first unit 1 and the second unit 2, or the second unit 2 and the third unit 3 is intermittent. Sensors and sensing functions that are used as feedback for controlling functional loads can be housed in at least one of the second unit 2 and the first unit 1. In one embodiment, the third unit 3 includes at least one stimulator. Thus, the second unit 2 may include a feedback control system that adjusts the function of the third unit 3 via power transfer. In one embodiment, the third unit 3 continuously receives power from the secondary power unit 2. In one embodiment, the third unit 3 receives power from the secondary power unit 2 intermittently only when stimulation or sensing is scheduled as directed by the first unit 1 or the second unit 2.

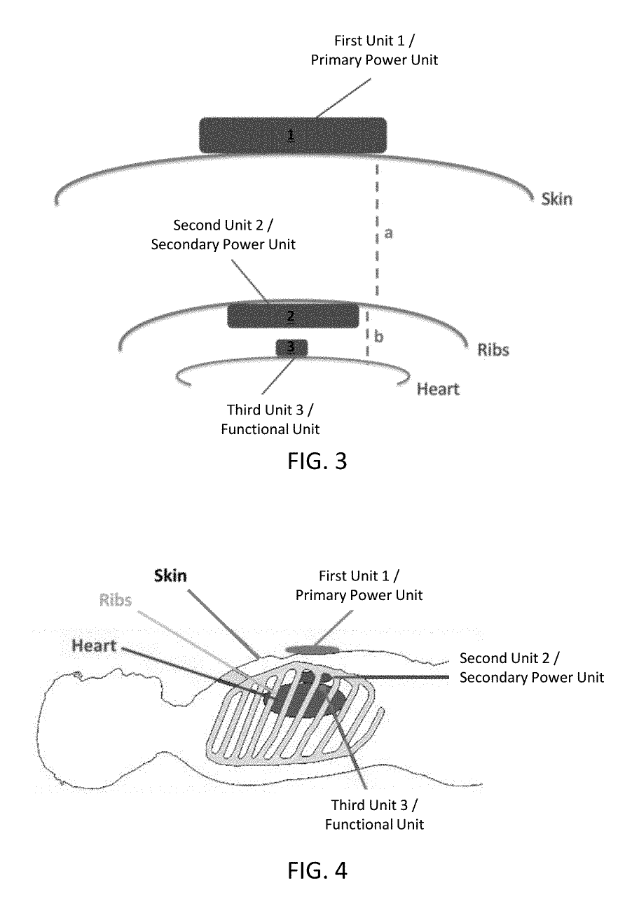

Now with reference to FIG. 3, in one embodiment, the primary power unit 1 is generally larger than the secondary power unit 2, which is in-turn larger than the implanted third unit 3. In one embodiment, the inductive coils of the primary power unit 1 are also larger than the inductive coils of the secondary power unit 2, which in-turn are larger than the inductive coils of the implanted third unit 3. In one embodiment, the distance “a” between the primary power unit 1 and the secondary power unit 2 is larger than the distance “b” between the secondary power unit 2 and the third unit 3.

In one embodiment, the described inductive power system is used to power a cardiac pacing system, in which the primary power unit 1 is located external (FIG. 4) or internal (FIG. 5) to the patient, the secondary power unit 2 is located in the thoracic cavity, and the third unit 3 is in contact with the heart from the outside or inside of the heart. In the preferred embodiment of such a pacing device, the primary component is placed external to the patient as a chest strap, and the patient wears the chest strap for a short period of time, for example during a yearly office visit. To charge the secondary power unit 2's rechargeable battery, the patient removes the chest strap following the recharging session until the subsequent cycle. The secondary power unit 2 is located substernal or subcostal and is implanted laparoscopically into the thoracic cavity, and the secondary power unit 2 contains the control system and logic circuitry that determines pacing activity based on cardiac electrical activity that is obtained wirelessly from a sensor in the third unit 3. The secondary power unit 2 also sends said data to a device external to the patient for monitoring of cardiac activity. The third unit 3 is located pericardially, for example, integrated into a stent placed into the coronary veins and implanted via catheter delivery, and placed in the external cardiac wall via stent integration decreases distance to the secondary power unit 2's inductive coil, decreasing risk of dislodgement inside the heart chambers, and improving fixation mechanism. The third unit 3 includes only an inductive coil receiver, capacitor, voltage rectifier IC, and sensing and pacing electrodes (i.e. no logic circuitry and no battery), thereby maintaining a versatile miniature size.

In other embodiments of the present invention, the described inductive power system is used to power functional loads of other implantable systems, including pressure sensors, temperature sensors, flow sensors, flow pumps, implantable cardioverter defibrillators, ECGs, deep brain stimulation devices, neuromodulators, and other monitoring, measuring, sensing, actuation, stimulation, and therapeutic devices.

The components of the system may function in any frequency band. However, in the preferred embodiment, the primary power unit 1 transmitter coil and secondary power unit 2 receiver coil communicate via a lower frequency, for example 2.4 MHz, to minimize heat dissipation to meet the Specific Absorption Rate (SAR) requirements set by the Federal Communications Commission (FCC), and the secondary power unit 2 transmitter coil and third unit 3 receiver coil communicate via a higher frequency, for example 433 MHz, since it is transmitting for a shorter period of time and over a shorter distance with less intervening tissue. Frequency ranges are not limited to the ranges of the exemplary embodiments disclosed herein. Due to the larger size of the first and second component, as described in the preferred embodiment, the coupling coefficient is larger and power transmission is more efficient despite the longer distance and lower functional frequency. The small size of the third unit 3, established to satisfy anatomical limitations, as described in the preferred embodiment, results in less efficient coupling and thus benefits from a higher frequency for sufficient power transfer.

Multiple implantations of the functional component (i.e. the third unit 3) may be present and communication may be established between the various components to allow for multiple functionalities. For example, one or more pacers can be positioned to allow for single or dual chamber pacing. Further, multiple secondary power units can be utilized between the primary power unit and the third unit. The coils in the multiple secondary power units can scale down stepwise moving away from the primary power unit and towards the third unit in certain multiple secondary power unit embodiments.

A method of wirelessly powering an implantable medical device is also described. Power is transferred from a primary power unit to a secondary power unit through induction at a first frequency. Power is also transferred from the secondary power unit to a third unit including the functional component of the medical device through induction at a second frequency. The first frequency is lower than the second frequency in one embodiment. The first frequency can be between 1.9 and 2.9 MHz, and in certain embodiments is substantially 2.4 MHz. The second frequency can be between 400 MHz and 466 MHz and in certain embodiments is substantially 433 MHz. Frequency ranges are not limited to the ranges of the exemplary embodiments disclosed herein. In one embodiment, the first and second frequency are substantially the same. In one embodiment, the primary power unit includes a first inductive coil, the secondary power unit includes a second and third inductive coil (or alternatively a single coil), and the third unit includes a fourth inductive coil. In one embodiment, the first inductive coil is larger than the second, third and fourth inductive coils. In one embodiment, the fourth inductive coil is smaller than the first, second and third inductive coils. The primary power unit can be attached to a patient's body, or subcutaneously implanted in the patient's body. The method can also include the steps of implanting the secondary power unit in a thoracic cavity of the patient and implanting the third unit in contact with the heart of a patient. In one embodiment, the sensory data is transmitted from the third unit to the secondary power unit and functional data can be transmitted from the secondary power unit to the third unit for adjusting the function of the medical device based on the functional data. Feedback data can also be transmitted from the third unit to at least one of the functional load, the primary power unit and the secondary power unit. In one embodiment, power is continuously transferred from the secondary power unit to the third unit. Alternately, power can be intermittently transferred from the secondary power unit to the third unit. In one embodiment, all communications, control and sensor components are stripped from the third unit. In one embodiment, sensing feedback including at least one of medical device function feedback, such as performance of the third unit, or patient physiological feedback, such as for example heart function, can be detected from a sensor at the second unit. In one embodiment, power can be transferred from the second unit to the third unit based on the sensed feedback.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

1. A wireless powering system for an implantable medical device comprising:
a first unit comprising a first power source electrically coupled to a first inductive coil;
a second unit comprising a first energy storage component electrically coupled to a second inductive coil; and
a third unit comprising a third inductive coil electrically coupled to a functional load.

2. The wireless powering system of claim 1, wherein the second unit further comprises a fourth inductive coil, wherein the first inductive coil is configured to transfer power to the second inductive coil at a first frequency, and the fourth inductive coil is configured to transfer power to the third inductive coil at a second frequency.

3. The wireless powering system of claim 2, wherein the first frequency is lower than the second frequency.

4. (canceled)

5. The wireless powering system of claim 1, wherein the first inductive coil is larger than the second, third and fourth inductive coils.

6. The wireless powering system of claim 5, wherein the third inductive coil is smaller than the first, second and fourth inductive coils.

7. (canceled)

8. The wireless powering system of claim 1, wherein the first unit is implantable.

9-12. (canceled)

13. The wireless powering system of claim 1, wherein the second unit further comprises a logic unit electrically coupled to the first energy storage component and configured to receive data from the third unit.

14. The wireless powering system of claim 13, wherein the logic unit is configured to transmit data to the third unit based on the received data.

15. The wireless powering system of claim 14, wherein the transmitted data comprises an instruction to adjust a function of the functional load.

16-18. (canceled)

19. The wireless powering system of claim 1, wherein the functional load is one of a sensor, electrode, actuator, motor and valve.

20. The wireless powering system of claim 1, wherein the functional load is one of a measuring device, sensing device, actuating device, stimulation device, therapeutic device, pacemaker, pressure sensor, temperature sensor, flow sensor, flow pump, implantable cardioverter defibrillator, ECG device, deep brain stimulator, and neuromodulator.

21-23. (canceled)

24. The wireless powering system of claim 1, wherein the third unit is configured to intermittently receive power from the second unit.

25-26. (canceled)

27. The wireless powering system of claim 1, wherein the third unit consists of a third inductive coil electrically coupled to a functional load.

28. A method of wirelessly powering an implantable medical device comprising:
transferring power from a first unit to a second unit through induction at a first frequency; and
transferring power from the second unit to a third unit through induction at a second frequency.

29. The method of claim 28, wherein the first frequency is lower than the second frequency.

30. (canceled)

31. The method of claim 28, wherein the first unit comprises a first inductive coil, the second unit comprises a second inductive coil, and the third unit comprises a third inductive coil.

32. The method of claim 28, wherein the first inductive coil is larger than the second and third inductive coils.

33. The method of claim 32, wherein the second unit comprises a fourth inductive coil, and wherein the third inductive coil is smaller than the first, second and fourth inductive coils.

34-41. (canceled)

42. The method of claim 28 further comprising:
intermittently transferring power from the second unit to the third unit.

43. The method of claim 28 further comprising:
sensing feedback comprising at least one of medical device function feedback or patient physiological feedback from a sensor at the second unit, and transferring power from the second unit to the third unit based on the sensed feedback.
